(19) United States
(12) Patent Application Publication
(10) Pub. No.: US 2023/0121726 A1
(43) Pub. Date: Apr. 20, 2023

(54) SEMICONDUCTOR TREATMENT LIQUID AND METHOD FOR MANUFACTURING SAME

(71) Applicant: TOKUYAMA CORPORATION, Yamaguchi (JP)

(72) Inventors: Masashi Shinagawa, Yamaguchi (JP); Takashi Tokunaga, Yamaguchi (JP); Yuu Mishima, Yamaguchi (JP); Shunsuke Hosaka, Yamaguchi (JP)

(73) Assignee: TOKUYAMA CORPORATION, Yamaguchi (JP)

(21) Appl. No.: 17/915,189

(22) PCT Filed: Mar. 30, 2021
(86) PCT No.: PCT/JP2021/013518

§ 371 Date: Sep. 28, 2022

(30) Foreign Application Priority Data
Apr. 2, 2020 (JP) 2020-067076

(51) Int. Cl. C11D 7/50; C11D 11/00; C11D 7/26; C07C 29/82

(52) U.S. Cl. CPC C11D 7/5022; C11D 11/0047; C11D 7/264; C07C 29/82

(57) ABSTRACT

Provided are: a semiconductor treatment liquid comprising high-purity isopropyl alcohol, wherein the concentration of the oxolane compound expressed in formula (1) below when held for 60 days in a nitrogen atmosphere at 50° C. in a SUS304 container is 25 ppb or less on a mass basis in relation to the isopropyl alcohol; and a method for manufacturing said semiconductor treatment liquid. In the formula, R¹ and R² each independently represent a hydrogen atom or a C1-3 alkyl group, and the total number of carbon atoms in R¹ and R² is 3 or less. R³ represents a hydrogen atom or an isopropyl group.

### TECHNICAL FIELD

The present invention relates to a semiconductor treatment liquid including high-purity isopropyl alcohol, and a method for manufacturing the same.

### BACKGROUND ART

Isopropyl alcohol (also referred to as 2-propanol) is an organic solvent used for various purposes, and is manufactured by e.g. a hydration method in which hydration of propylene is carried out.

Isopropyl alcohol is commonly manufactured in a petrochemical complex which can provide propylene as a raw material, transferred to an area of demand after manufacturing, and stored in a storage tank. As described above, isopropyl alcohol is frequently stored over a long period of time from manufacture to use. Therefore, an increase in impurities in isopropyl alcohol in storage for a long period of time is a severe problem.

In particular, when isopropyl alcohol having increased impurities by being stored for a long period of time is used to clean electronic devices such as semiconductor devices, residues derived from impurities in isopropyl alcohol may remain on the surface of electronic devices after cleaning and drying.

Patent Document 1, for example, describes that organic impurities dissolved in isopropyl alcohol cohere with vaporization of isopropyl alcohol to become relatively large particles, which remain in an object to be treated to generate particulate contamination (particulate defects).

As described above, residues after cleaning and drying are a factor to generate defects in electronic devices. It is therefore desired that the concentration of organic impurities in isopropyl alcohol used as a cleaning liquid, particularly the concentration of high-boiling impurities having higher boiling points than that of isopropyl alcohol, which high-boiling impurities become residues after treatment, be reduced as much as possible. In addition, when low-boiling impurities having lower boiling points than that of isopropyl alcohol exist, there is also a possibility that high-boiling impurities will be generated by the progress of various reactions in a container during storage for a long period of time. Therefore, isopropyl alcohol, in which even when isopropyl alcohol is stored for a long period of time, organic impurities causing residues after cleaning and drying do not increase, has been desired.

About an increase in impurities during the storage of isopropyl alcohol, Patent Document 2, for example, describes that by allowing electron donors to peroxy radicals generated by the oxidation reaction of isopropyl alcohol to exist in isopropyl alcohol, the progress of oxidative degradation can be highly suppressed, and ketone generated during the storage of isopropyl alcohol can be significantly reduced.

In addition, Patent Document 3 describes that high-boiling impurities having higher boiling points than that of isopropyl alcohol are removed by distilling isopropyl alcohol. Patent Document 3 also describes that in combination with removal of high-boiling impurities, low-boiling impurities having lower boiling points than that of isopropyl alcohol are removed by distillation. In addition, Patent Document 3 suggests that in the operation of semiconductor manufacture, these organic impurities in isopropyl alcohol remain in wafers and cause defects.
Patent Document 1: Japanese Unexamined Patent Application, Publication No. 2016-004902
Patent Document 2: Japanese Unexamined Patent Application, Publication No. 2016-179956
Patent Document 3: Japanese Unexamined Patent Application (Translation of PCT Application), Publication No. 2003-535836

### DISCLOSURE OF THE INVENTION

### Problems to be Solved by the Invention

Patent Document 3, however, does not reveal specific types of high-boiling impurities and low-boiling impurities, and does not indicate how these impurities act to each other to cause defects in the above semiconductor applications. Therefore, organic impurities are removed by a common distillation method, and only general quality as isopropyl alcohol is obtained. As a result, the total amount of organic impurities is high, 200 to 500 ppm (see paragraph [0018]).

The present inventors investigated and found that in some impurities, an increase in the concentration thereof cannot be suppressed only by allowing electron donors to peroxy radicals to exist in isopropyl alcohol. It was particularly found that the concentration of organic impurities could be increased during transport and storage even when isopropyl alcohol was manufactured and the quality control thereof at the time of shipping was carried out so as to satisfy a control value of 50 ppb or less (on a mass basis) as the concentration of impurities at a boiling point of 120° C. or higher, required as isopropyl alcohol for the electronic industry.

The present inventors further investigated and found that in the above-described organic impurities, an oxolane compound represented by Formula (1) below and generated by the condensation of an α,β-unsaturated aldehyde compound and an alcohol existed, and this oxolane compound increased with time during storage.

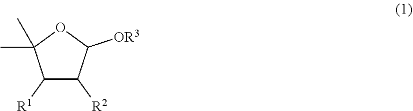

In Formula (1), R¹ and R² each independently represent a hydrogen atom or a C1-C3 alkyl group, with the proviso that the total number of carbon atoms in R¹ and R² is 3 or less. R³ represents a hydrogen atom or an isopropyl group.

A subject of the present invention is to provide a semiconductor treatment liquid including high-purity isopropyl alcohol, which has excellent long-term storage stability, wherein the concentration of an oxolane compound as impurities is low and an increase in the concentration of this oxolane compound with time is suppressed, and a method for manufacturing the same.

### Means for Solving the Problems

The present inventors diligently investigated to solve the above problems. As a result, the present inventors found that the above problems could be solved not only by directly reducing an oxolane compound included as impurities in isopropyl alcohol (composition), but also controlling the concentration of an α,β-unsaturated aldehyde compound represented by Formula (2) below within equal to or less than a specific amount, thereby completing the present invention. The α,β-unsaturated aldehyde compound represented by Formula (2) below is considered to be changed into an oxolane compound during storage by some influence. An increase in the oxolane compound with time can be suppressed by reducing these impurities and it is possible to obtain isopropyl alcohol in which the concentration of the oxolane compound is maintained low.

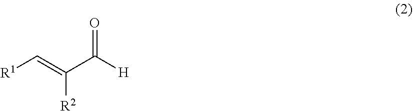

In Formula (2), R¹ and R² have the same meanings as in Formula (1) above.

Conventionally, organic impurities having higher boiling points than that of isopropyl alcohol have been considered to be removed by a distillation step of removing high-boiling impurities, and the separation of high-boiling impurities, which are not separated by common industrial processes, has been considered difficult due to a high affinity for isopropyl alcohol. Therefore, when isopropyl alcohol is used to clean electronic devices, an inevitable amount of organic impurities is considered to remain in an object to be treated. Furthermore, it was also found that when isopropyl alcohol was held in a sealed container such as a canister can or a transfer tank container and stored for a long period of time, residues of such organic impurities increased. This phenomenon significantly occurs when the above sealed container is made of resin such as polyolefin resin or fluorine resin or glass, and intensively occurs when it is made of metal such as stainless steel, HASTELLOY, Inconel or Monel, and especially remarkably occurs particularly when it is made of stainless steel, particularly SUS304.

In such circumstances, the present inventors succeeded in highly reducing the concentration of an oxolane compound and also highly reducing a causative substance to generate an oxolane compound during the storage of the isopropyl alcohol by highly removing high-boiling impurities. As a result, isopropyl alcohol, in which the concentration of the oxolane compound can be maintained to a low level, 25 ppb or less on a mass basis, even after an acceleration test which is presumed as long-term storage, was found for the first time.

The following embodiments are included in specific means to solve the above problems.

<1> A semiconductor treatment liquid including high-purity isopropyl alcohol,

in which a concentration of an oxolane compound is 25 ppb or less on a mass basis with respect to the isopropyl alcohol, the oxolane compound being represented by Formula (1) below:

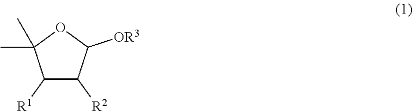

in which R¹ and R² each independently represent a hydrogen atom or a C1-C3 alkyl group, with the proviso that the total number of carbon atoms in R¹ and R² is 3 or less, and R³ represents a hydrogen atom or an isopropyl group, provided that the concentration of the oxolane compound is a concentration measured after the semiconductor treatment liquid is stored at 50° C. in a nitrogen atmosphere in a SUS304 container for 60 days.

<2> The semiconductor treatment liquid as described in <1>, in which the total number of carbon atoms in R¹ and R² in Formula (1) above is 1 to 3.

<3> The semiconductor treatment liquid as described in <1>, in which the oxolane compound represented by Formula (1) above is 4,5,5-trimethyltetrahydrofuran-2-ol or 2-isopropoxy-4,5,5-trimethyltetrahydrofuran.

<4> A semiconductor treatment liquid including high-purity isopropyl alcohol, containing:

an α,β-unsaturated aldehyde compound represented by Formula (2) below:

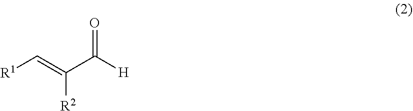

in which R¹ and R² each independently represent a hydrogen atom or a C1-C3 alkyl group, with the proviso that the total number of carbon atoms in R¹ and R² is 3 or less,
in which the total concentration of the α,β-unsaturated aldehyde compound represented by Formula (2) above and an oxolane compound represented by Formula (1) below:

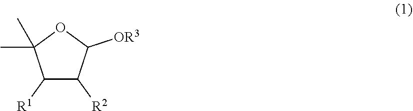

in which R¹ and R² have the same meanings as in Formula (2) above and R³ is a hydrogen atom or an isopropyl group, is 25 ppb or less on a mass basis with respect to the isopropyl alcohol, provided that the concentration of the α,β-unsaturated aldehyde compound is a concentration obtained by converting the concentration of the α,β-unsaturated aldehyde compound represented by Formula (2) above into a concentration of the oxolane compound represented by Formula (1) above in which R³ is an isopropyl group and which is derived from the α,β-unsaturated aldehyde compound.

<5> The semiconductor treatment liquid as described in <4>, in which the number of carbon atoms in the α,β-unsaturated aldehyde compound represented by Formula (2) above is 4 to 6.

<6> The semiconductor treatment liquid as described in <4>, in which the α,β-unsaturated aldehyde compound represented by Formula (2) above is crotonaldehyde.

<7> The semiconductor treatment liquid as described in any one of <1> to <6>, in which the amount of water is 0.1 to 100 ppm on a mass basis.

<8> The semiconductor treatment liquid as described in any one of <1> to <7>, in which isopropyl alcohol is obtained by direct hydration of propylene.

<9> A method for manufacturing a semiconductor treatment liquid as described in any one of <1> to <7>, the method including:

a low-boiling distillation step of distilling a crude isopropyl alcohol aqueous solution with a water content of 80 mass % or more in a low-boiling distillation column, to distill off low-boiling impurities having lower boiling points than that of isopropyl alcohol from the column top of the low-boiling distillation column, and also to obtain an isopropyl alcohol aqueous solution in which low-boiling impurities have been removed, from the column bottom of the low-boiling distillation column,
an azeotropic distillation step of distilling the isopropyl alcohol aqueous solution in an azeotropic distillation column, to distill off an azeotropic mixture of isopropyl alcohol and water from the column top of the azeotropic distillation column, and also to discharge high-boiling impurities having higher boiling points than that of isopropyl alcohol from the column bottom of the azeotropic distillation column, and a dehydration step of dehydrating the azeotropic mixture to obtain high-purity isopropyl alcohol,
in which a liquid flowing downward inside the low-boiling distillation column is taken as a side stream from the middle of the low-boiling distillation column in a proportion of 0.1 vol % or more with respect to the crude isopropyl alcohol aqueous solution fed to the low-boiling distillation column, to discharge substantially the entire amount of the side stream outside the system in the low-boiling distillation step.

<10> The method for manufacturing a semiconductor treatment liquid as described in <9>, in which a place to take the side stream in the low-boiling distillation step is a place at 10 to 50% from the top plate in the low-boiling distillation column.

<11> The method for manufacturing a semiconductor treatment liquid as described in <9> or <10>, in which the crude isopropyl alcohol aqueous solution is obtained by direct hydration of propylene.

### Effects of the Invention

According to the present invention, it is possible to provide a semiconductor treatment liquid including high-purity isopropyl alcohol, which has excellent long-term storage stability, wherein the concentration of an oxolane compound as impurities is low, and an increase in the concentration of this oxolane compound with time is suppressed, and a method for manufacturing the same.

The oxolane compound has a higher boiling point than that of isopropyl alcohol, and thus when isopropyl alcohol including the oxolane compound is used as a cleaning liquid for electronic devices, it can be a cause of residues after cleaning and drying. In this respect, the semiconductor treatment liquid of the present invention has an extremely low concentration of the oxolane compound, and moreover this concentration is maintained low, and thus the liquid can be suitably used as a cleaning liquid for semiconductor manufacturing processes.

### BRIEF DESCRIPTION OF THE DRAWINGS

FIG. 1 is a process diagram which shows a typical method for manufacturing high-purity isopropyl alcohol to manufacture a semiconductor treatment liquid.

### PREFERRED MODE FOR CARRYING OUT THE INVENTION

The embodiments of the present invention will now be described in detail. In the following description, “%”, “ppm” and “ppb” representing concentrations are on a mass basis in all cases including examples.

### <Semiconductor Treatment Liquid>

The semiconductor treatment liquid according to the present embodiment is a semiconductor treatment liquid including high-purity isopropyl alcohol, wherein after the semiconductor treatment liquid is stored at 50° C. in a nitrogen atmosphere in a SUS304 container for 60 days, the concentration of an oxolane compound represented by Formula (1) below is maintained low, 25 ppb or less on a mass basis with respect to isopropyl alcohol.

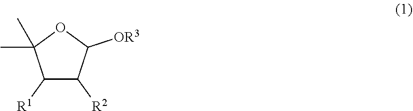

In Formula (1), R¹ and R² each independently represent a hydrogen atom or a C1-C3 alkyl group, with the proviso that the total number of carbon atoms in R¹ and R² is 3 or less. R³ represents a hydrogen atom or an isopropyl group.

The concentration of the oxolane compound represented by Formula (1) above and the concentration of an α,β-unsaturated aldehyde compound described below are concentrations when the concentration of isopropyl alcohol in high-purity isopropyl alcohol is used as a criterion. In addition, the amount of water described below is an amount when the total amount of high-purity isopropyl alcohol is used as a criterion. These concentrations or amount is measured by measurement methods described below.

It should be noted that the high-purity isopropyl alcohol in the present embodiment means that in which the concentration of isopropyl alcohol is 99.99% or more and preferably 99.999% or more when represented as a concentration not including water by mass spectrometry using gas chromatography (GC/MS).

### (Impurities; Oxolane Compound)

The oxolane compound in the present embodiment is a compound represented by Formula (1) above, and most of the compounds are generated by the condensation of an α,β-unsaturated aldehyde compound represented by Formula (2) below with an alcohol under a catalyst. The oxolane compound having 7 carbon atoms, for example, is generated from crotonaldehyde and isopropyl alcohol.

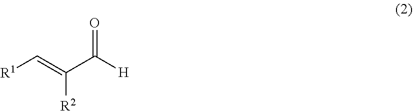

In Formula (2), R¹ and R² have the same meanings as in Formula (1) above.

Here, among α,β-unsaturated aldehyde compounds represented by Formula (2) above, compounds having 4 or more carbon atoms have higher boiling points than that of isopropyl alcohol, and are difficult to remove by purification using common distillation. Meanwhile, compounds having 7 or more carbon atoms have much higher boiling points than that of isopropyl alcohol, and can be removed to an extent by purification using common distillation. Therefore, the effect of the present invention is more remarkably displayed by removing α,β-unsaturated aldehyde compounds having 4 to 6 carbon atoms. From these viewpoints and because the amount included in isopropyl alcohol is high, among α,β-unsaturated aldehyde compounds, crotonaldehyde is most typical.

In Formula (1) above, R¹ and R² each independently represent a hydrogen atom or a C1-C3 alkyl group. In addition, R³ represents a hydrogen atom or an isopropyl group. Examples of the C1-C3 alkyl group include a methyl group, an ethyl group, an n-propyl group, an isopropyl group and the like. The total number of carbon atoms in R¹ and R² is 3 or less. In addition, from the above reason, the total number of carbon atoms in R¹ and R² is preferably 1 or more.

Examples of the oxolane compound represented by Formula (1) above are shown in Table 1 below.

TABLE 1

| Number of | | | | |
|---|---|---|---|---|
| carbon | | | | |
| atoms in | | | | |
| oxolane | | | | |
| compound | R¹ | R² | R³ | Compound name |
| 6 | Hydrogen | Hydrogen | Hydrogen | 5,5-Dimethyltetrahydrofuran-2- |
| | atom | atom | atom | ol |
| 7 | Methyl | Hydrogen | Hydrogen | 4,5,5- |
| | group | atom | atom | Trimethyltetrahydrofuran-2-ol |
| | Hydrogen | Methyl | Hydrogen | 3,5,5- |
| | atom | group | atom | Trimethyltetrahydrofuran-2-ol |
| 8 | Methyl | Methyl | Hydrogen | 3,4,5,5- |
| | group | group | atom | Tetramethyltetrahydrofuran-2- |
| | Ethyl | Hydrogen | Hydrogen | 4-Ethyl-5,5- |
| | group | atom | atom | dimethyltetrahydrofuran-2-ol |
| | Hydrogen | Ethyl | Hydrogen | 3-Ethyl-5,5- |
| | atom | group | atom | dimethyltetrahydrofuran-2-ol |
| 9 | Hydrogen | Hydrogen | Isopropyl | 2-Isopropoxy-5,5- |
| | atom | atom | group | dimethyltetrahydrofuran |
| | Ethyl | Methyl | Hydrogen | 4-Ethyl-3,5,5- |
| | group | group | atom | trimethyltetrahydrofuran-2-ol |
| | Methyl | Ethyl | Hydrogen | 3-Ethyl-4,5,5- |
| | group | group | atom | trimethyltetrahydrofuran-2-ol |
| | n-Propyl | Hydrogen | Hydrogen | 4-Propyl-5,5- |
| | group | atom | atom | dimethyltetrahydrofuran-2-ol |
| | Hydrogen | n-Propyl | Hydrogen | 3-Propyl-5,5- |
| | atom | group | atom | dimethyltetrahydrofuran-2-ol |
| | Isopropyl | Hydrogen | Hydrogen | 4-Isopropyl-5,5- |
| | group | atom | atom | dimethyltetrahydrofuran-2-ol |
| | Hydrogen | Isopropyl | Hydrogen | 3-Isopropyl-5,5- |
| | atom | group | atom | dimethyltetrahydrofuran-2-ol |
| 10 | Methyl | Hydrogen | Isopropyl | 2-Isopropoxy-4,5,5- |
| | group | atom | group | trimethyltetrahydrofuran |
| | Hydrogen | Methyl | Isopropyl | 2-Isopropoxy-3,5,5- |
| | atom | group | group | trimethyltetrahydrofuran |
| 11 | Ethyl | Hydrogen | Isopropyl | 2-Isopropoxy-4-ethyl-5,5- |
| | group | atom | group | dimethyltetrahydrofuran |
| | Hydrogen | Ethyl | Isopropyl | 2-Isopropoxy-3-ethyl-5,5- |
| | atom | group | group | dimethyltetrahydrofuran |
| | Methyl | Methyl | Isopropyl | 2-Isopropoxy-3,4,5,5- |
| | group | group | group | tetramethyltetrahydrofuran |
| 12 | Ethyl | Methyl | Isopropyl | 2-Isopropoxy-4-ethyl-3,5,5- |
| | group | group | group | trimethyltetrahydrofuran |
| | Methyl | Ethyl | Isopropyl | 2-Isopropoxy-3-ethyl-4,5,5- |
| | group | group | group | trimethyltetrahydrofuran |
| | n-Propyl | Hydrogen | Isopropyl | 2-Isopropoxy-4-propyl-5,5- |
| | group | atom | group | dimethyltetrahydrofuran |
| | Hydrogen | n-Propyl | Isopropyl | 2-Isopropoxy-3-propyl-5,5- |
| | atom | group | group | dimethyltetrahydrofuran |
| | Isopropyl | Hydrogen | Isopropyl | 2-Isopropoxy-4-isopropyl-5,5- |
| | group | atom | group | dimethyltetrahydrofuran |
| | Hydrogen | Isopropyl | Isopropyl | 2-Isopropoxy-3-isopropyl-5,5- |
| | atom | group | group | dimethyltetrahydrofuran |

Among the oxolane compounds represented by Formula (1) above, the concentration of a compound wherein R¹ is a methyl group, R² is a hydrogen atom, and R³ is a hydrogen atom or an isopropyl group derived from crotonaldehyde, i.e. 4,5,5-trimethyltetrahydrofuran-2-ol and 2-isopropoxy-4,5,5-trimethyltetrahydrofuran, is preferably reduced.

The high-purity isopropyl alcohol in the present embodiment is used as a semiconductor treatment liquid, and thus it is demanded that the concentration of the oxolane compound represented by Formula (1) above be 25 ppb or less at the time of use. The concentration of the oxolane compound represented by Formula (1) above at the time of use is preferably 10 ppb or less and more preferably 2 ppb or less. When the high-purity isopropyl alcohol in the present embodiment is used as a cleaning liquid in semiconductor manufacturing processes, in order that residues will not remain in an object to be treated after cleaning and drying, a smaller amount of the oxolane compound having a higher boiling point than that of isopropyl alcohol (i.e. nearer to 0 ppb) is more preferred. However, the lower limit of the concentration of the oxolane compound is preferably 0.1 ppb or more and more preferably 0.3 ppb or more in view of the industrial manufacture, storage and transport of isopropyl alcohol.

In the high-purity isopropyl alcohol in the present embodiment, the concentration of the oxolane compound represented by Formula (1) above can be highly reduced, and the concentration of the oxolane compound can be usually reduced to 5 ppb or less when it is immediately after manufacture and to 1 ppb or less when it is a good compound. In addition to this, a causative substance to generate the oxolane compound during the storage thereof can be also highly reduced, and even when high-purity isopropyl alcohol is stored at 50° C. in a nitrogen atmosphere in a SUS304 container for 60 days (hereinafter this acceleration test is simply referred to as “storage test”), the concentration of the above oxolane compound can be maintained to a desired low value, i.e. 25 ppb or less, preferably 10 ppb or less and more preferably 2 ppb or less. When it is used to clean electronic devices such as semiconductor devices, defects due to residues can be largely improved by properties in which an increase in the oxolane compound is suppressed even after such storage test.

The storage test and the measurement of the concentration of the oxolane compound are specifically carried out by the following methods. That is, 3 L of high-purity isopropyl alcohol is put in a container of 20 L capacity made of SUS304, and deoxidation is carried out by providing nitrogen into the liquid at 2 L/min for 30 minutes. After deoxidation, the container is sealed so that oxygen will not enter, and the container is stored in a 50° C. thermostatic bath for 60 days. After completion of the storage test, the concentration of the oxolane compound in isopropyl alcohol in the container is measured by gas chromatography-mass spectrometry (GC-MS). It should be noted that SUS304 is typical as a material of a container for a semiconductor treatment liquid including high-purity isopropyl alcohol such as a canister can or transport tank container, and is a material which particularly remarkably shows a phenomenon of an increase in the oxolane compound during storage as described above.

Properties which do not increase the oxolane compound in isopropyl alcohol even by such long-term and high temperature storage test are caused by highly reducing the concentration of an α,β-unsaturated aldehyde compound represented by Formula (2) above. That is, such α,β-unsaturated aldehyde compound is inevitably contained at the time of manufacturing isopropyl alcohol, and because the compound is contained, the oxolane compound is considered to increase with time after completion of the manufacture of isopropyl alcohol. Therefore, the properties in the above storage test are satisfied by highly reducing these specific impurities.

### (Impurities; α,β-Unsaturated Aldehyde Compound)

In the present embodiment, specific examples of the α,β-unsaturated aldehyde compound represented by Formula (2) above included in high-purity isopropyl alcohol include crotonaldehyde, methacrolein, 2-pentenal, ethacrolein, 2-methyl-2-butenal, 2-ethyl-2-butenal, 2-methyl-2-pentenal, 2-hexenal, 2-methylenepentanal, 4-methyl-2-pentenal, 2-isopropylacrolein and the like. When the α,β-unsaturated aldehyde compound has cis-trans isomers, the cis-form and trans-form are also included.

Examples of factors why the α,β-unsaturated aldehyde compound represented by Formula (2) above is included in isopropyl alcohol can include “impurities included in propylene/acetone as a material for isopropyl alcohol”, “a byproduct in the synthesis reaction of isopropyl alcohol”, “an alcohol compound included in isopropyl alcohol after manufacture” and the like. Due to these factors, commonly, the α,β-unsaturated aldehyde compound represented by Formula (2) above is inevitably contaminated in industrially manufactured isopropyl alcohol.

As described above, the α,β-unsaturated aldehyde compound represented by Formula (2) above is impurities generated as a byproduct of reactions, and also by e.g. a reaction step, a purification step and an oxidative reaction during storage. Because it is included in isopropyl alcohol in high amounts, the range of the concentration has not been strictly controlled until now.

According to investigations by the present inventors, however, it is considered that for example when an α,β-unsaturated aldehyde compound is included in isopropyl alcohol, isopropyl alcohol and the α,β-unsaturated aldehyde compound react as shown in the reaction formula below, and an oxolane compound is derived and increases with time. It should be noted that the reaction formula below shows an example of the oxolane compound represented by Formula (1) above wherein R¹ is a methyl group, R² is a hydrogen atom and R³ is an isopropyl group.

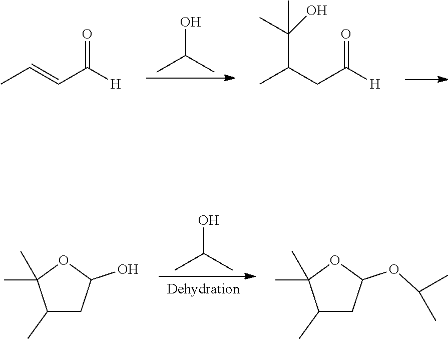

According to the reaction formula above, an increase in the oxolane compound derived from crotonaldehyde can be suppressed by controlling the concentration of crotonaldehyde included in isopropyl alcohol.

In addition to crotonaldehyde, α,β-unsaturated aldehyde compounds having different numbers of carbon atoms are included in isopropyl alcohol, and oxolane compounds are also derived from the α,β-unsaturated aldehyde compounds other than crotonaldehyde. In the reaction of acrolein having 3 carbon atoms and isopropyl alcohol, for example, the oxolane compound represented by the formula below increases with time.

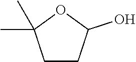

Therefore, it is presumed that an increase in the oxolane compound with time can be suppressed by controlling the concentration of α,β-unsaturated aldehyde compounds included in isopropyl alcohol within a specific range.

The concentration of α,β-unsaturated aldehyde compounds represented by Formula (2) above included in high-purity isopropyl alcohol is preferably controlled to satisfy the following requirements in view of a low concentration of the oxolane compound when it is used as a semiconductor treatment liquid and the suppression of an increase in the oxolane compound during storage. That is, the total concentration of an α,β-unsaturated aldehyde compound represented by Formula (2) above, and an oxolane compound represented by Formula (1) above is controlled to preferably 25 ppb or less (more preferably 10 ppb or less and further preferably 2 ppb or less) on a mass basis with respect to isopropyl alcohol when the concentration of the α,β-unsaturated aldehyde compound represented by Formula (2) above is converted into the concentration of the oxolane compound wherein R³ in Formula (1) above is an isopropyl group, derived from the α,β-unsaturated aldehyde compound.

When an α,β-unsaturated aldehyde compound represented by Formula (2) above is changed into an oxolane compound represented by Formula (1) above, the molecular weight thereof increases. However, the degree is about 2 to 2.5 times even in a compound wherein an isopropyl group is introduced into R³ in Formula (1) above. In addition, all α,β-unsaturated aldehyde compounds represented by Formula (2) above are not changed into oxolane compounds represented by Formula (1) above. The reaction rates in the above storage test are, for example, normally 70% or less, and 50% or less in many cases. As described above, therefore, the concentration of the oxolane compound can be reduced to an extremely small amount immediately after manufacturing high-purity isopropyl alcohol. Even if a system in which almost all the total concentration of an α,β-unsaturated aldehyde compound and an oxolane compound is occupied by the α,β-unsaturated aldehyde compound is considered, as long as the concentration of the α,β-unsaturated aldehyde compound is 10 ppb or less, preferably 5 ppb or less and more preferably 1 ppb or less, the system satisfies the above range provided by the total concentration.

The lower limit of the concentration of an α,β-unsaturated aldehyde compound is preferably 0 ppb because it is considered that the generation of an oxolane compound can be suppressed by a lower concentration thereof. However, in view of industrial manufacture of isopropyl alcohol, the lower limit is preferably 0.01 ppb, more preferably 0.1 ppb and further preferably 0.5 ppb.

It should be noted that an organic substance having a high boiling point may be generated during storage by the condensation of α,β-unsaturated aldehyde compounds included as impurities with each other, and these condensed substances may also become residues after cleaning and drying. Therefore, the condensation of α,β-unsaturated aldehyde compounds with each other can be also prevented by controlling the concentration of the α,β-unsaturated aldehyde compounds within a specific range.

### (Other Impurities)

The high-purity isopropyl alcohol in the present embodiment may include other impurities which are inevitably contaminated in the manufacture thereof. Examples of impurities inevitably contaminated include water, free acids, organic impurities, inorganic impurities and the like. Among these, the organic impurities are organic impurities which are not separated in the step of distilling isopropyl alcohol and are contaminated.

### (Water)

In the high-purity isopropyl alcohol in the present embodiment, the amount of water is preferably 0.1 to 100 ppm. The water in isopropyl alcohol is considered to cause residues after cleaning and drying and watermark, and there is also a risk that it acts as a catalyst. Therefore, the amount of water is preferably 100 ppm or less. Meanwhile, a reaction to generate an oxolane compound is a dehydration reaction, and thus the existence of water in isopropyl alcohol is considered to be able to further suppress the generation of an oxolane compound in view of chemical equilibrium. Therefore, the amount of water is preferably 0.1 ppm or more. From the viewpoint that high-purity isopropyl alcohol is used as a semiconductor treatment liquid and the generation of an oxolane compound is suppressed, the amount of water is more preferably 1 to 50 ppm and further preferably 3 to 25 ppm.

Furthermore, it is preferable that the mass of an α,β-unsaturated aldehyde compound represented by Formula (2) above included in the high-purity isopropyl alcohol in the present embodiment and the amount of water satisfy the following relationship. Specifically, a rate p represented by Formula (I) below is preferably 0.00002 to 0.01 and more preferably 0.0001 to 0.001.

p=(mass of α,β-unsaturated aldehyde compound)/(amount of water) (I)

As described above, a reaction to generate an oxolane compound is a dehydration reaction, and water existing in isopropyl alcohol is considered to be able to suppress the generation of an oxolane compound in view of chemical equilibrium. It is therefore considered that when the above rate p is above 0.01, there is a tendency that water is reduced and the generation of an oxolane compound increases. Meanwhile, when the rate p is less than 0.00002, an α,β-unsaturated aldehyde compound tends to increase, and finally there is a risk that an oxolane compound will increase.

From the above reasons, it is considered that in the high-purity isopropyl alcohol in the present embodiment, the generation of an oxolane compound can be further suppressed by controlling the above rate p within a range of 0.00002 to 0.01.

The high-purity isopropyl alcohol in the present embodiment has much more excellent storage stability by controlling the amount of water as well, and can be transported and stored for a long period of time. Then it can be suitably used, for example, as a cleaning liquid in semiconductor manufacture processes.

### (Other Impurities: Free Acid)

Free acids are presumed to be inevitably contaminated in the manufacture of high-purity isopropyl alcohol and act as catalysts for the generation of an oxolane compound. Therefore, the concentration of free acids in the high-purity isopropyl alcohol in the present embodiment is preferably 10 ppm or less, more preferably 100 ppb or less and further preferably 10 ppb or less. A smaller lower limit is more preferred; however, the lower limit is commonly 0.1 ppb or more in view of industrial manufacture, storage and transport.

### <Method for Manufacturing High-Purity Isopropyl Alcohol>

The high-purity isopropyl alcohol in the present embodiment may be one manufactured in any method as long as the above-described properties are satisfied. The high-purity isopropyl alcohol is manufactured, for example, by a reaction step of obtaining a crude isopropyl alcohol aqueous solution by direct hydration of propylene, and a purification step of obtaining high-purity isopropyl alcohol by purifying the crude isopropyl alcohol aqueous solution.

### [Reaction Step]

The direct hydration of propylene in the reaction step is represented by the following formula. The following reaction is carried out in a reactor to obtain a reaction mixture.

C₃H₆+H₂O→CH₃CH(OH)CH₃

In the reaction step, it is preferable that the reaction pressure is 150 to 250 atm, and the reaction temperature is 200 to 300° C. In addition, in the reaction step, acid catalysts of various polyanions such as molybdenum-based and tungsten-based inorganic ion exchangers can be used. Among acid catalysts, at least one selected from the group consisting of phosphotungstic acid, silicotungstic acid and silicomolybdic acid is preferred from the viewpoint of reaction activity. The selectivity of isopropyl alcohol, a reaction product, can be increased by adopting such reaction conditions, and isopropyl alcohol, in which impurities, particularly e.g. organic acids, high-boiling compounds having 4 or more carbon atoms, an α,β-unsaturated aldehyde compound represented by Formula (2) above and an oxolane compound represented by Formula (1) above, are reduced, can be obtained.

A reaction mixture including isopropyl alcohol generated by the above reaction is taken from the reactor in a state of being dissolved in the water phase. Unreacted propylene dissolved in the water phase is separated as a gas by reducing the pressure and temperature to recover a reaction product. The separated propylene is used as raw material again.

### [Purification Step]

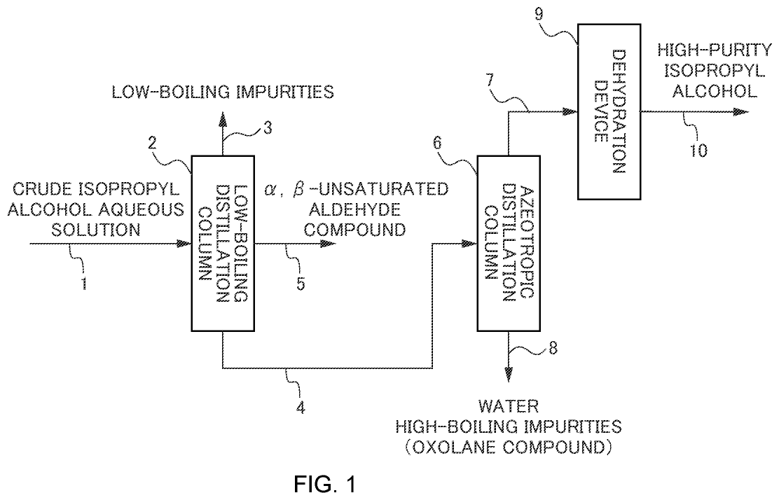

A crude isopropyl alcohol aqueous solution with a water content of 80% or more is commonly obtained by the above reaction step. In this purification step, this crude isopropyl alcohol aqueous solution is purified to obtain high-purity isopropyl alcohol. This purification step preferably includes a low-boiling distillation step of distilling a crude isopropyl alcohol aqueous solution with a water content of 80% or more in the low-boiling distillation column, to distill off low-boiling impurities having lower boiling points than that of isopropyl alcohol from the column top of the low-boiling distillation column, and also to obtain an isopropyl alcohol aqueous solution in which low-boiling impurities have been removed, from the column bottom of the low-boiling distillation column; an azeotropic distillation step of distilling the isopropyl alcohol aqueous solution in an azeotropic distillation column, to distill off an azeotropic mixture of isopropyl alcohol and water from the column top of the azeotropic distillation column, and also to discharge high-boiling impurities having higher boiling points than that of isopropyl alcohol from the column bottom of the azeotropic distillation column; and a dehydration step of dehydrating the azeotropic mixture to obtain high-purity isopropyl alcohol. In particular, it is preferable that a liquid flowing downward inside the low-boiling distillation column is taken as a side stream from the middle of the low-boiling distillation column in a proportion of 0.1 vol % or more with respect to the crude isopropyl alcohol aqueous solution fed to the low-boiling distillation column, to discharge substantially the total amount of the side stream outside the system in the low-boiling distillation step. The outline of this purification step is shown in the process diagram in FIG. 1.

### (Low-Boiling Distillation Step)

In the low-boiling distillation step, low-boiling impurities having lower boiling points than that of isopropyl alcohol are distilled off from the column top of the low-boiling distillation column, and also an isopropyl alcohol aqueous solution in which low-boiling impurities have been removed is obtained from the column bottom of the low-boiling distillation column.

In FIG. 1, for example, the crude isopropyl alcohol aqueous solution obtained in the reaction step is fed to the low-boiling distillation column 2 through the conduit 1 and distilled. Because of this, low-boiling impurities having lower boiling points than that of isopropyl alcohol (olefins such as ethylene and propylene; aldehydes such as acetaldehyde and propylene aldehyde; etc.) are distilled off from the conduit 3 in the column top. Meanwhile, the isopropyl alcohol aqueous solution in which low-boiling impurities have been removed is discharged from the conduit 4 in the column bottom.

It should be noted that a constant amount of oxolane compound is contained in the crude isopropyl alcohol aqueous solution; however, the compound has a higher boiling point than that of isopropyl alcohol (e.g. in the case of 4,5,5-trimethyltetrahydrofuran-2-ol, boiling point: 184° C.), and is thus contained in the isopropyl alcohol aqueous solution flowing in the conduit 4.

In order to highly reduce the concentration of α,β-unsaturated aldehyde compounds in high-purity isopropyl alcohol, it is important that in the low-boiling distillation step, a liquid flowing downward inside the low-boiling distillation column is taken as a side stream from the middle of the low-boiling distillation column in a proportion of 0.1 vol % or more with respect to the crude isopropyl alcohol aqueous solution fed to the low-boiling distillation column, to discharge substantially the total amount of the side stream outside the system. In FIG. 1, specifically, the side stream is taken in the above-described amount from the middle of the low-boiling distillation column 2 toward the conduit 5 to dispose of substantially the total amount thereof outside the system.

Here, it is known that when manufacturing high-purity isopropyl alcohol, low-boiling impurities are removed by distilling the crude isopropyl alcohol aqueous solution in the low-boiling distillation column as described in e.g. Patent Document 3, etc. At this time, however, taking the side stream in the middle of the low-boiling distillation column is not generally carried out because the yield of isopropyl alcohol drops.

Meanwhile, the present inventors found for the first time the above oxolane compound as a causative substance which is a factor to generate defects in electronic devices when using high-purity isopropyl alcohol as a semiconductor treatment liquid, and moreover found a unique phenomenon, in which the oxolane compound is generated from an α,β-unsaturated aldehyde compound as a precursor not only in a manufacturing step of isopropyl alcohol but also during the storage thereof. As a result of investigating various behaviors of the α,β-unsaturated aldehyde compound, an original method was found, in which the side stream is taken in the middle of the low-boiling distillation column to dispose of substantially the total amount thereof.

The boiling point of α,β-unsaturated aldehyde compounds is 53° C. in acrolein having 3 carbon atoms, which is lower than 82.5° C., the boiling point of isopropyl alcohol, and in the case of 4 or more carbon atoms, the boiling point is high, 104° C., in e.g. trans-crotonaldehyde, which is above the boiling point of isopropyl alcohol. Therefore, as with the oxolane compound, the α,β-unsaturated aldehyde compounds having such high boiling points must be intrinsically contained in the isopropyl alcohol aqueous solution which is discharged from the column bottom in low-boiling distillation.

If this is correct, naturally discharging such high-boiling impurities from the column bottom in the azeotropic distillation step, which is a later step, to remove will be a common method. However, as with water, the α,β-unsaturated aldehyde compounds are azeotroped with isopropyl alcohol, and thus are difficult to highly remove by discharge from the column bottom. In addition, even if isopropyl alcohol is distilled after dehydration, because the α,β-unsaturated aldehyde compounds are azeotroped with isopropyl alcohol, they cannot be removed as high-boiling impurities or low-boiling impurities, and it is difficult to highly reduce the α,β-unsaturated aldehyde compounds. Therefore, high-purity isopropyl alcohol, in which the concentration of the α,β-unsaturated aldehyde compounds is reduced to satisfy a value provided in the present embodiment, has not been obtained by conventional manufacturing methods.

In such circumstances, the present inventors found that by taking a side stream from the middle part of the low-boiling distillation column and disposing of it, even the α,β-unsaturated aldehyde compounds having high boiling points could be highly removed. This is presumed to be caused by a fact that due to an affinity for other low-boiling impurities (or bad compatibility in an isopropyl alcohol aqueous solution), the α,β-unsaturated aldehyde compounds having high boiling points flow upward with them inside the column and are condensed in the middle part. This is an unexpected behavior for those skilled in the art.

In the low-boiling distillation step, the amount of the side stream taken from the middle of the low-boiling distillation column is a proportion of 0.1 vol % or more with respect to the crude isopropyl alcohol aqueous solution fed to the low-boiling distillation column, preferably 0.1 to 1.0 vol % and more preferably 0.15 to 0.30 vol %. When this amount taken is lower than 0.1 vol %, the effect of removing the α,β-unsaturated aldehyde compounds tends to be insufficient. In addition, when the amount taken is excessively high, loss of isopropyl alcohol is greater and efficiency is reduced.

The side stream may be taken continuously or intermittently during distillation, but preferably taken continuously. When it is taken continuously, the amount taken is obtained as the amount per minute of side stream taken during distillation with respect to the amount per minute of crude isopropyl alcohol fed to the distillation column.

The total amount of the side stream taken from the middle of the low-boiling distillation column is preferably disposed of from the viewpoint of enhancing properties of removing α,β-unsaturated aldehyde compounds; however, as long as the amount thereof is small to an extent that the effect of removal is not lost, even when the side stream is circulated in the low-boiling distillation column, substantially the total amount thereof is considered to be disposed of. Specifically, even if the side stream is circulated in the low-boiling distillation column in 10 mass % or less and more preferably 1 mass % or less with respect to the flow taken from the middle of the low-boiling distillation column, that is accepted in the present embodiment.

The low-boiling distillation column may be any of shelf-type and packed column-type, but is preferably the shelf-type. The number of plates in the shelf-type or the number of plates corresponding to the distillation columns converted into a plate column are not restricted. However, costs for distillation equipment increase when the numbers are too many, and a reduction of the α,β-unsaturated aldehyde compounds is insufficient when the numbers are too small. Therefore, the number of plates is preferably 10 to 100, more preferably 15 to 80 and further preferably 20 to 50. As the plates of the shelf-type column, e.g. a cross-flow tray and a shower tray can be used. Examples of substances packed in the packed column type include known substances packed such as Raschig ring and Lessing ring. The material for the column and the material for substances packed are not restricted, and iron, SUS, HASTELLOY, borosilicate glass, fused quartz, fluorine resin (e.g. polytetrafluoroethylene), etc. can be used.

In the low-boiling distillation step, a site to take the side stream in the low-boiling distillation column is not particularly restricted as long as the site is in the middle part of the low-boiling distillation column; however, from the viewpoint of high properties of removing α,β-unsaturated aldehyde compounds, it is preferably a 10 to 50% place from the top plate in the low-boiling distillation column and more preferably a 15 to 40% place. In the distillation column having 100 plates, for example, the side stream is preferably taken at a place from 10th to 50th plates from the top. When the place to take the side stream is not the above place, the concentration of α,β-unsaturated aldehydes is not sufficiently high, and the effect of reducing α,β-unsaturated aldehyde compounds is small. In addition, the side stream may be taken from one site or two or more sites in the middle of the low-boiling distillation column. When it is taken from two or more sites, each site is preferably in the above range.

The site to feed the crude isopropyl alcohol aqueous solution to the low-boiling distillation column may be any place from the column bottom to column top; however, it is preferably fed from the middle part. It is more preferably fed to a 10 to 50% place from the top plate in the low-boiling distillation column.

The reflux ratio of distillates from the column top of the low-boiling distillation column is not restricted. However, when it is too high, the low-boiling distillation column becomes larger and equipment costs and operation costs increase, and when it is too small, the yield of isopropyl alcohol decreases. Therefore, it is preferably 10 to 50000, more preferably 50 to 2000 and further preferably 100 to 1000.

The pressure in the distillation column is not particularly restricted; however, it is preferably from ordinary pressure to slightly pressurized state, 0.1 to 0.15 MPa (absolute pressure), due to ease of operation. The temperature of column top and column bottom may be appropriately set depending on the above pressure.

### (Azeotropic Distillation Step)

In the azeotropic distillation step, the isopropyl alcohol aqueous solution discharged from the column bottom in the low-boiling distillation step is distilled in the azeotropic distillation column to distill off an azeotropic mixture of isopropyl alcohol and water from the column top of the azeotropic distillation column, and also to discharge high-boiling impurities having higher boiling points than that of isopropyl alcohol from the column bottom of the azeotropic distillation column.

In FIG. 1, for example, the isopropyl alcohol aqueous solution discharged from the column bottom of the low-boiling distillation column 2 is fed through the conduit 4 to the azeotropic distillation column 6 and distilled. The azeotropic temperature of isopropyl alcohol and water is 80.1° C., and an azeotropic mixture (amount of water: about 12%) of isopropyl alcohol and water is distilled off from the conduit 7 in the column top by distilling the above isopropyl alcohol aqueous solution at the temperature. In the column bottom, meanwhile, high-boiling impurities are discharged with water from the conduit 8. At this time, the oxolane compound contained in the isopropyl alcohol aqueous solution is also highly removed as one of high-boiling impurities discharged from the column bottom. As a result, the amount of the oxolane compound can satisfy the above desired regulation and can be reduced in the high-purity isopropyl alcohol finally obtained.

Furthermore, the distillation in the azeotropic distillation step may be carried out in accordance with various conditions described in the low-boiling distillation step.

### (Dehydration Step)

In the dehydration step, the azeotropic mixture obtained in the azeotropic distillation step is dehydrated to obtain high-purity isopropyl alcohol. In FIG. 1, for example, the azeotropic mixture obtained in the azeotropic distillation step is fed to the dehydration device 9 through the conduit 7 and dehydrated. A high-purity isopropyl alcohol aqueous solution in which water has been removed is discharged from the conduit 10.

The dehydration method in the dehydration step is not particularly restricted, and distillation, adsorption, membrane permeation, etc. are used. When dehydration distillation is carried out, water can be removed by adding e.g. diethyl ether, benzene, trichloroethylene and dichloromethane to make three-component azeotropic composition.

The high-purity isopropyl alcohol obtained by dehydration may be further purified by a method such as distillation or adsorption as needed. In addition, metal and inorganic particles may be removed by filter filtration, and metal ions may be removed by an ion exchange resin column. It can be more advantageously used as a semiconductor treatment liquid by removing impurities other than organic compounds as described above.

The high-purity isopropyl alcohol obtained as above is held in a sealed container such as a canister can or tank container and transported to a consuming region. In particular, when the material of the sealed container is made of metal such as stainless steel, HASTELLOY, Inconel or Monel, a semiconductor treatment liquid has a low amount of oxolane compound and significantly displays an excellent effect of suppressing defects. This is particularly remarkable when stainless steel, particularly SUS304, is used.

When high-purity isopropyl alcohol is held in a sealed container, storage stability can be further increased by filling the space in the container with an inert gas such as nitrogen gas. In addition, the sealed container after transport is preferably enclosed with an inert gas such as nitrogen gas or argon gas.

In the high-purity isopropyl alcohol in the present embodiment, causative substances, which are a factor to generate defects in electronic devices, are reduced. Therefore, it is useful when used as a semiconductor treatment liquid. It is specifically useful as a cleaning liquid for electronic devices, a rinse solution, a draining agent, a developer, etc. and particularly useful as a cleaning liquid.

### EXAMPLES

The present invention will now be described in more detail by way of examples thereof. It should be noted, however, that the present invention is not limited to these examples.

First, the analysis and quantification method of impurities, etc. will be described.

### [Method for Measuring Concentration of Oxolane Compound]

The concentration of an oxolane compound represented by Formula (1) above included in isopropyl alcohol was measured using GC-MS in measurement conditions described below. The detected oxolane compound was compared with the peak area of a standard substance quantitated previously to quantitate the concentration of the detected oxolane compound by selected ion monitoring (SIM).

### —Measurement Conditions—

Device: 7890B/5977B (manufactured by Agilent Technologies Japan, Ltd.)
Analysis column: CPWAX52CB (60 m×0.5 mm×0.50 μm)
Column temperature: 30° C. (held for 3 min)→elevated at 5° C./min→100° C.→elevated at 10° C./min→240° C. (held for 6 min)
Carrier gas: helium
Carrier gas flow rate: 2 mL/min
Injection port temperature: 240° C.
Sample injection: pulsed splitless
Injection pulse pressure: 90 psi (2 min)
Split vent flow rate: 50 mL/min (2 min)
Gas saver: 20 mL/min (5 min)
Transfer line temperature: 240° C.
Ion source, quadrupole temperature: 230° C., 150° C.
—SIM monitor ion—
m/z: 69, 113, 115

### [Method 1 for Measuring Concentration of α,β-Unsaturated Aldehyde Compound]

The quantitative analysis of α,β-unsaturated aldehyde compounds represented by Formula (2) above included in isopropyl alcohol was carried out using GC/MS by selected ion monitoring (SIM) in measurement conditions described below. The lower limit of quantitation was calculated using the standard substances of α,β-unsaturated aldehyde compounds. As a result, the lower limit of quantitation of acrolein, trans-crotonaldehyde, trans-2-pentenal and trans-2-hexenal was 5 ppb.

### —Measurement Conditions—

Device: GC-2010 plus/QP2010 ultra (manufactured by SHIMADZU CORPORATION)
Analysis column: CPWAX52CB (60 m×0.5 mm×0.50 μm)
Column temperature: 75° C.
Carrier gas: helium
Carrier gas flow rate: 1 mL/min
Injection port temperature: 150° C.
Sample injection: split
Split ratio: 1:5
Transfer line temperature: 230° C.
Ion source, quadrupole temperature: 200° C.
Scan ions: m/z=30-300
—SIM monitor ion—
m/z: 56 (acrolein analysis)
m/z: 70 (crotonaldehyde analysis)
m/z: 84 (2-pentenal analysis)
m/z: 83 (2-hexenal analysis)

### [Method 2 for Measuring Concentration of α,β-Unsaturated Aldehyde Compound]

The lower limit of quantitation in the above-described method for measuring the concentration of an α,β-unsaturated aldehyde compound is 5 ppb. Therefore when the concentration of an α,β-unsaturated aldehyde compound in isopropyl alcohol is 5 ppb or less, the α,β-unsaturated aldehyde compound was derivatized with 2,4-dinitrophenylhydrazine (DNPH) in the following method and then condensed, and the α,β-unsaturated aldehyde compound was then quantitated.

That is, 100 mg of 2,4-dinitrophenylhydrazine (DNPH) and 100 mL of 2 mol/L hydrochloric acid were mixed to prepare a DNPH hydrochloric acid solution. Isopropyl alcohol, 50 mL, and 1 mL of the DNPH hydrochloric acid solution were mixed, and a sample was air-dried with nitrogen at 1 L/min for about 3 hours for 50-fold condensation to make 1 mL. The obtained condensed sample was analyzed by high performance liquid chromatography (HPLC) in the following conditions. The lower limit of quantitation was calculated using the standard substances of α,β-unsaturated aldehyde compounds. As a result, the lower limit of quantitation of acrolein, trans-crotonaldehyde, trans-2-pentenal and trans-2-hexenal was 0.1 ppb.

### —Measurement Conditions—

Device: Ultimate 3000 (manufactured by Thermo Fishier Scientific)
Column: Inertsil ODS-2 (manufactured by GL Sciences Inc.)
Column packing particle size: 5 μm
Column diameter: 2.1 mm
Column length: 250 mm
Flow rate: 0.2 ml/min
Column temperature: 40° C.

### Detector: UV (360 nm)

Sample injection amount: 8 μL
Mobile phase ratio: 0→14 min:acetonitrile/1 mM acetic acid+2 mM ammonium acetate=48/52 (constant), 14 min→25 min:acetonitrile/1 mM acetic acid+2 mM ammonium acetate=48/52→100/0 (gradient), 25 min→45 min:acetonitrile/1 mM acetic acid+2 mM ammonium acetate=100/0 (constant).

### [Method for Measuring Amount of Water]

Device: Karl Fischer Moisture Meter AQ-7 (manufactured by HIRANUMA Co., Ltd.)
Method: 0.8 g of a measurement sample was taken by Terumo Syringe in Glovebox at a dew point of −80° C. or lower and measured by Karl Fischer Moisture Meter.

### Example 1

### [Production of Crude Isopropyl Alcohol]

Propylene as a raw material, including 40000 ppm propane, 20 ppm ethane, 8 ppm butene, 0.1 ppm or less pentene and 0.1 ppm or less hexene as impurities, was prepared. In addition, water as a raw material having a pH of 3.0 adjusted by adding phosphotungstic acid, an acid catalyst, was prepared. In a reactor of 10 L capacity, water the temperature of which had been raised to 110° C. was added at a feed rate of 18.4 kg/h (20 L/h because the density is 920 kg/m³) and propylene was also added at a feed rate of 1.2 kg/h.

In the reactor, the reaction temperature was 280° C. and the reaction pressure was 250 atm. Propylene and water were allowed to react to obtain a crude isopropyl alcohol aqueous solution. The generated reaction product including isopropyl alcohol was cooled to 140° C. and the pressure was reduced to 18 atm to recover propylene dissolved in water included in the crude isopropyl alcohol aqueous solution as a gas. The recovered propylene was put in a propylene recovery drum for reuse as a raw material. At this time, the rate of conversion of the fed propylene was 84.0%, and the selectivity of propylene to isopropyl alcohol was 99.2%.

### [Purification Operation]

### (Low-Boiling Distillation Step)

A 10 L flask was put in an oil bath, and an Oldershaw distillation column having 20 plates was set. From the second plate from the top plate in the distillation column, the crude isopropyl alcohol aqueous solution was fed at 10 L/h. The percentage of water content was 95% in this crude isopropyl alcohol. Distillation was carried out with an oil bath of 120° C. at a column top temperature of 75 to 85° C. and a column pressure (gauge pressure) of 0 to 10 kPa. The reflux ratio was 100, the side stream was discharged outside the system from the third plate from the top plate in the distillation column at 17 mL/h (0.17 vol % with respect to the crude isopropyl alcohol aqueous solution fed to the distillation column), and the liquid was sent to the next step at about 10 L/h so that the amount of liquid in the 10 L flask would be maintained to about 5 L.

### (Azeotropic Distillation Step)

Subsequently, 10 L of the isopropyl alcohol aqueous solution discharged from the 10 L flask was put in another 10 L flask, which was put in an oil bath. The oil bath temperature was 120° C. and the temperature of the upper part of the flask was 75 to 85° C. for heating. Vapor distilled off was cooled by Liebig condenser through which about 25° C. water had flown to obtain the condensed isopropyl alcohol aqueous solution. The condensed isopropyl alcohol has azeotropic composition with water, and the percentage of water content was 12%. Meanwhile, the bottom liquid in the 10 L flask was discharged outside the system.

### (Dehydration Step)

In a 10 L flask, 3 L of isopropyl alcohol of azeotropic composition with water obtained in the azeotropic distillation step, and 7 L of benzene were put, and this was put in an oil bath. The oil bath temperature was 90° C. and the temperature of the upper part of the flask was 65 to 75° C. for heating. The generated vapor including water and benzene was cooled by Liebig condenser through which about 25° C. water had flown to recover water and benzene, and dehydrated high-purity isopropyl alcohol was obtained in the flask.

The concentration of α,β-unsaturated aldehyde compounds in the obtained high-purity isopropyl alcohol was measured, and acrolein, crotonaldehyde, 2-pentenal and 2-hexenal were detected. The total concentration of these α,β-unsaturated aldehyde compounds was about 1 ppb. In addition, the concentration of the oxolane compound was 0.1 ppb or less. It should be noted that the amount of moisture was 12 ppm in the obtained high-purity isopropyl alcohol, and the concentration of isopropyl alcohol was 99.999% or more when represented as a concentration not including water.

### [Storage Test]

The storage test was then carried out in conditions described below to confirm the storage stability of the high-purity isopropyl alcohol obtained by the above production method.

In a 20 L SUS304 container, 3 L of high-purity isopropyl alcohol was put, and nitrogen was fed at 1 L/min for 30 minutes to carry out deoxidation. After deoxidation, the container was sealed so that oxygen would not enter. The sealed container was stored in a 50° C. drier for 60 days. After completion of the storage test, when measured in accordance with the above-described method for measuring an oxolane compound, the concentration of the oxolane compound was 1 ppb (Table 2).

As described above, in high-purity isopropyl alcohol, in which the total concentration of acrolein, crotonaldehyde, 2-pentenal and 2-hexenal was reduced to about 1 ppb, the concentration of the oxolane compound was low, 1 ppb, even after the storage test, and it could be confirmed to have very excellent long-term storage stability.

### Example 2

High-purity isopropyl alcohol was obtained in the same manner as in Example 1 except that in the method for producing high-purity isopropyl alcohol in Example 1, the site to take the side stream from the distillation column in the low-boiling distillation step was changed to the seventh plate from the top plate in the distillation column. In the obtained high-purity isopropyl alcohol, the amount of water was 15 ppm and the concentration of isopropyl alcohol was 99.999% or more when represented as a concentration not including water.

As shown in Table 2, the total concentration of the α,β-unsaturated aldehyde compounds was about 1 ppb in the obtained high-purity isopropyl alcohol. In addition, the concentration of the oxolane compound was 0.1 ppb or less, and was low, 1 ppb, even after the storage test. Because of this, this high-purity isopropyl alcohol could be confirmed to have very excellent long-term storage stability.

### Example 3

High-purity isopropyl alcohol was obtained in the same manner as in Example 1 except that in the method for producing high-purity isopropyl alcohol in Example 1, the amount of the side stream taken from the distillation column in the low-boiling distillation step was changed to 12 mL/h. In the obtained high-purity isopropyl alcohol, the amount of water was 13 ppm and the concentration of isopropyl alcohol was 99.999% or more when represented as a concentration not including water.

As shown in Table 2, the total concentration of the α,β-unsaturated aldehyde compounds was about 4 ppb in the obtained high-purity isopropyl alcohol. In addition, the concentration of the oxolane compound was 0.1 ppb or less, and was low, 2 ppb, even after the storage test. Because of this, this high-purity isopropyl alcohol could be confirmed to have excellent long-term storage stability.

### Example 4

High-purity isopropyl alcohol was obtained in the same manner as in Example 3 except that in the method for producing high-purity isopropyl alcohol in Example 3, the site to take the side stream from the distillation column in the low-boiling distillation step was changed to the seventh plate from the top plate in the distillation column. In the obtained high-purity isopropyl alcohol, the amount of water was 14 ppm and the concentration of isopropyl alcohol was 99.999% or more when represented as a concentration not including water.

As shown in Table 2, the total concentration of the α,β-unsaturated aldehyde compounds was about 5 ppb in the obtained high-purity isopropyl alcohol. In addition, the concentration of the oxolane compound was 0.1 ppb or less, and was low, 4 ppb, even after the storage test. Because of this, this high-purity isopropyl alcohol could be confirmed to have excellent long-term storage stability.

### Example 5

High-purity isopropyl alcohol was obtained in the same manner carried out as in Example 3 except that in the method for producing high-purity isopropyl alcohol in Example 3, the site to take the side stream from the distillation column in the low-boiling distillation step was changed to the eleventh plate from the top plate in the distillation column. In the obtained high-purity isopropyl alcohol, the amount of water was 15 ppm and the concentration of isopropyl alcohol was 99.999% or more when represented as a concentration not including water.

As shown in Table 2, the total concentration of the α,β-unsaturated aldehyde compounds was about 9 ppb in the obtained high-purity isopropyl alcohol. In addition, the concentration of the oxolane compound was 0.1 ppb or less, and was low, 8 ppb, even after the storage test. Because of this, this high-purity isopropyl alcohol could be confirmed to have excellent long-term storage stability.

### Comparative Example 1

High-purity isopropyl alcohol was obtained in the same manner as in Example 1 except that in the method for producing high-purity isopropyl alcohol in Example 1, the reflux ratio in the low-boiling distillation step was total reflux, and the side stream was not taken from the distillation column. In the obtained high-purity isopropyl alcohol, the amount of water was 12 ppm and the concentration of isopropyl alcohol was 99.999% or more when represented as a concentration not including water.

As shown in Table 2, the total concentration of the α,β-unsaturated aldehyde compounds was about 38 ppb in the obtained high-purity isopropyl alcohol. In addition, the concentration of the oxolane compound was 0.1 ppb or less; however, the concentration was largely increased to 35 ppb after the storage test.

### Comparative Example 2

High-purity isopropyl alcohol was obtained in the same manner carried out as in Example 1 except that in the method for producing high-purity isopropyl alcohol in Example 1, the amount of the side stream taken from the distillation column in the low-boiling distillation step was changed to 5 mL/h. In the obtained high-purity isopropyl alcohol, the amount of water was 16 ppm and the concentration of isopropyl alcohol was 99.999% or more when represented as a concentration not including water.

As shown in Table 2, the total concentration of the α,β-unsaturated aldehyde compounds was about 30 ppb in the obtained high-purity isopropyl alcohol. In addition, the concentration of the oxolane compound was 0.1 ppb or less; however, the concentration was largely increased to 28 ppb after the storage test.

TABLE 2

| | | Number of plates | Amount of side stream taken | Water |
|---|---|---|---|---|
| | Reflux | to take | (mL/h) | amount |
| | ratio | side stream | (Proportion of side stream taken*¹) | (ppm) |
| Example 1 | 100 | 3 | 17 | 12 |
| | | | (0.17 vol %) | |
| Example 2 | 100 | 7 | 17 | 15 |
| | | | (0.17 vol %) | |
| Example 3 | 100 | 3 | 12 | 13 |
| | | | (0.12 vol %) | |
| Example 4 | 100 | 7 | 12 | 14 |
| | | | (0.12 vol %) | |
| Example 5 | 100 | 11 | 12 | 14 |
| | | | (0.12 vol %) | |
| Comparative | Total | No taken | | 12 |
| Example 1 | reflux | | | |
| Comparative | 100 | 3 | 5 | 16 |
| Example 2 | | | (0.05 vol %) | |

| | | | | | | Oxolane |
|---|---|---|---|---|---|---|
| | α,β-Unsaturated aldehyde compounds (ppb) | | | | | compound |
| | (Conversion concentration of oxolane compound | | | | Oxolane | after |
| | (R³: isopropyl group)) | | | | compound | storage test |
| | Acrolein | Crotonaldehyde | 2-Pentenal | 2-Hexenal | (ppb) | (ppb) |
| Example 1 | 0.1 or less | 2 | 0.1 or less | 0.1 or less | 0.1 or less | 1 |
| | (0.28 or less) | (2.2) | (0.20 or less) | (0.18 or less) | | |
| Example 2 | 0.1 or less | 1 | 0.1 or less | 0.1 or less | 0.1 or less | 1 |
| | (0.28 or less) | (2.2) | (0.20 or less) | (0.18 or less) | | |
| Example 3 | 0.1 or less | 4 | 0.1 or less | 0.1 or less | 0.1 or less | 2 |
| | (0.28 or less) | (8.8) | (0.20 or less) | (0.18 or less) | | |
| Example 4 | 0.1 or less | 5 | 0.1 or less | 0.1 or less | 0.1 or less | 4 |
| | (0.28 or less) | (11) | (0.20 or less) | (0.18 or less) | | |
| Example 5 | 0.1 or less | 9 | 0.1 or less | 0.1 or less | 0.1 or less | 8 |
| | (0.28 or less) | (20) | (0.20 or less) | (0.18 or less) | | |
| Comparative | 0.1 or less | 38 | 0.1 or less | 0.1 or less | 0.1 or less | 35 |
| Example 1 | (0.28 or less) | (84) | (0.20 or less) | (0.18 or less) | | |
| Comparative | 0.1 or less | 30 | 0.1 or less | 0.1 or less | 0.1 or less | 28 |
| Example 2 | (0.28 or less) | (66) | (0.20 or less) | (0.18 or less) | | |

*¹Proportion of side stream taken to crude isopropyl alcohol aqueous solution fed to distillation column

### EXPLANATION OF REFERENCE NUMERALS

1, 3, 4, 5, 7, 8, 10 Conduit
2 Low-boiling distillation column
6 Azeotropic distillation column
9 Dehydration device

1. A semiconductor treatment liquid comprising high-purity isopropyl alcohol,
wherein a concentration of an oxolane compound is 25 ppb or less on a mass basis with respect to the isopropyl alcohol, the oxolane compound being represented by Formula (1) below: 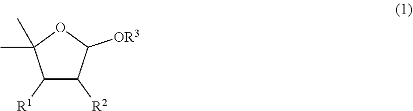
wherein R¹ and R² each independently represent a hydrogen atom or a C1-C3 alkyl group, with a proviso that a total number of carbon atoms in R¹ and R² is 3 or less, and R³ represents a hydrogen atom or an isopropyl group,
provided that the concentration of the oxolane compound is a concentration measured after the semiconductor treatment liquid is stored at 50° C. in a nitrogen atmosphere in a SUS304 container for 60 days.

2. The semiconductor treatment liquid according to claim 1, wherein the total number of carbon atoms in R¹ and R² in the Formula (1) is 1 to 3.

3. The semiconductor treatment liquid according to claim 1, wherein the oxolane compound represented by the Formula (1) is 4,5,5-trimethyltetrahydrofuran-2-ol or 2-isopropoxy-4,5,5-trimethyltetrahydrofuran.

4. A semiconductor treatment liquid comprising high-purity isopropyl alcohol, comprising:
an α,β-unsaturated aldehyde compound represented by Formula (2) below: 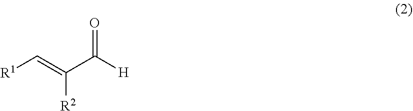
wherein R¹ and R² each independently represent a hydrogen atom or a C1-C3 alkyl group, with a proviso that a total number of carbon atoms in R¹ and R² is 3 or less,
wherein a total concentration of the α,β-unsaturated aldehyde compound represented by the Formula (2) and an oxolane compound represented by Formula (1) below: 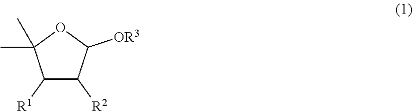
wherein R¹ and R² have the same meanings as in the Formula (2) and R³ represents a hydrogen atom or an isopropyl group,
is 25 ppb or less on a mass basis with respect to the isopropyl alcohol, provided that the concentration of the α,β-unsaturated aldehyde compound is a concentration obtained by converting the concentration of the α,β-unsaturated aldehyde compound represented by the Formula (2) into a concentration of the oxolane compound represented by the Formula (1) in which R³ is an isopropyl group and which is derived from the α,β-unsaturated aldehyde compound.

5. The semiconductor treatment liquid according to claim 4, wherein a number of carbon atoms in the α,β-unsaturated aldehyde compound represented by the Formula (2) is 4 to 6.

6. The semiconductor treatment liquid according to claim 4, wherein the α,β-unsaturated aldehyde compound represented by the Formula (2) is crotonaldehyde.

7. The semiconductor treatment liquid according to claim 1, wherein an amount of water is 0.1 to 100 ppm on a mass basis.

8. The semiconductor treatment liquid according to claim 1, wherein the isopropyl alcohol is obtained by direct hydration of propylene.

9. A method for manufacturing a semiconductor treatment liquid according to claim 1, the method comprising:
a low-boiling distillation step of distilling a crude isopropyl alcohol aqueous solution with a water content of 80 mass % or more in a low-boiling distillation column, to distill off low-boiling impurities having lower boiling points than that of isopropyl alcohol from a column top of the low-boiling distillation column, and also to obtain an isopropyl alcohol aqueous solution in which the low-boiling impurities have been removed, from a column bottom of the low-boiling distillation column,
an azeotropic distillation step of distilling the isopropyl alcohol aqueous solution in an azeotropic distillation column, to distill off an azeotropic mixture of isopropyl alcohol and water from a column top of the azeotropic distillation column, and also to discharge high-boiling impurities with higher boiling points than that of isopropyl alcohol from a column bottom of the azeotropic distillation column, and
a dehydration step of dehydrating the azeotropic mixture to obtain high-purity isopropyl alcohol,
in the low-boiling distillation step, a liquid flowing downward inside the low-boiling distillation column being taken as a side stream from a middle of the low-boiling distillation column in a proportion of 0.1 vol % or more with respect to the crude isopropyl alcohol aqueous solution fed to the low-boiling distillation column, to discharge substantially an entire amount of the side stream outside a system.

10. The method for manufacturing a semiconductor treatment liquid according to claim 9, wherein a place to take the side stream in the low-boiling distillation step is a place at 10 to 50% from a top plate of the low-boiling distillation column.

11. The method for manufacturing a semiconductor treatment liquid according to claim 9, wherein the crude isopropyl alcohol aqueous solution is obtained by direct hydration of propylene.

12. The semiconductor treatment liquid according to claim 4, wherein an amount of water is 0.1 to 100 ppm on a mass basis.

13. The semiconductor treatment liquid according to claim 4, wherein the isopropyl alcohol is obtained by direct hydration of propylene.

14. A method for manufacturing a semiconductor treatment liquid according to claim 4, the method comprising:
a low-boiling distillation step of distilling a crude isopropyl alcohol aqueous solution with a water content of 80 mass % or more in a low-boiling distillation column, to distill off low-boiling impurities having lower boiling points than that of isopropyl alcohol from a column top of the low-boiling distillation column, and also to obtain an isopropyl alcohol aqueous solution in which the low-boiling impurities have been removed, from a column bottom of the low-boiling distillation column,
an azeotropic distillation step of distilling the isopropyl alcohol aqueous solution in an azeotropic distillation column, to distill off an azeotropic mixture of isopropyl alcohol and water from a column top of the azeotropic distillation column, and also to discharge high-boiling impurities with higher boiling points than that of isopropyl alcohol from a column bottom of the azeotropic distillation column, and
a dehydration step of dehydrating the azeotropic mixture to obtain high-purity isopropyl alcohol,
in the low-boiling distillation step, a liquid flowing downward inside the low-boiling distillation column being taken as a side stream from a middle of the low-boiling distillation column in a proportion of 0.1 vol % or more with respect to the crude isopropyl alcohol aqueous solution fed to the low-boiling distillation column, to discharge substantially an entire amount of the side stream outside a system.

15. The method for manufacturing a semiconductor treatment liquid according to claim 14, wherein a place to take the side stream in the low-boiling distillation step is a place at 10 to 50% from a top plate of the low-boiling distillation column.

16. The method for manufacturing a semiconductor treatment liquid according to claim 14, wherein the crude isopropyl alcohol aqueous solution is obtained by direct hydration of propylene.
